(19) United States
(12) Patent Application Publication
(10) Pub. No.: US 2002/0197184 A1
(43) Pub. Date: Dec. 26, 2002

(54) System and method for sterilizing a continuously moving web for forming, filling, and sealing flexible packages

(76) Inventor: Sevugan Palaniappan, Sugar Land, TX (US)

Correspondence Address: FINNEGAN, HENDERSON, FARABOW, GARRETT &, DUNNER LLP, 1300 I STREET, NW, WASHINGTON, DC, 20006, US

(21) Appl. No.: 09/884,907

(22) Filed: Jun. 21, 2001

(51) Int. Cl. A61L 2/00; A61L 2/08; A61L 9/00; A62B 7/08; B01J 19/08

(52) U.S. Cl. 422/22; 422/24; 422/27; 422/121; 422/186.05; 422/186.07; 422/292; 422/300; 422/305

(57) ABSTRACT

A sterilization system for use with a form, fill, seal machine to sterilize the web material being processed by the machine. The web passes seriatim through an ionized air wash, a chemical pre-wash, a sterilant bath, squeeze rollers, hot air knives and a UV radiation source. The synergism of the respective in-line treatments facilitates efficient sterilization at high web feed rates.

### BACKGROUND OF THE INVENTION

1. Field of the Invention

The present invention relates to a sterilization system and method for a web of material being fed to a form, fill and seal packaging machine. More specifically, the present invention relates to a sterilization system and method, which achieves high sterilization efficiencies for high-speed web feed rates into the machine.

2. Description of Related Art

Heretofore, the forming, sealing and filling of flexible packages, and particularly aseptic packages, have been performed by various techniques and apparatus. But, due to the nature of the packages to be filled and formed, and the aseptic environmental requirements, conventional systems have not been able to produce aseptic shelf-stable products at high speeds.

It is desirable to achieve above 300 packages/min, preferably above 600 packs/min, 80 to 85% efficiency of the filling line, but heretofore such speeds have not been achievable. Accordingly, a need in the art exists for a system and method for continuously forming, sealing and filling flexible packages at increased rates of speed as compared to prior techniques and systems.

However, the faster the rate of speed, the achievement of satisfactory sterilization levels becomes more problematic, since the residence time of the web at any sterilization station decreases with increased feed rates.

### SUMMARY OF THE INVENTION

Accordingly, it is a primary aspect of the present invention to provide a sterilization system and method having increased sterilization efficiencies, thus permitting use in high-speed form, fill and seal packaging machines.

It is a primary goal of the present invention to achieve up to a cumulative 5-log reduction of highly resistant spores, such as Bacillus subtilis, or other microorganisms on the web for aseptic use due to the synergies of the combined sterilization steps.

The aspects of the present invention are generally fulfilled by providing an apparatus for sterilizing web material being fed to a package form-fill-seal machine comprising:

a first bath of liquid chemical, such as an acid solution for pre-washing the web passing therethrough;

a second bath of liquid sterilant for sterilizing the web passing therethrough;

a cleaning device for removing excess sterilant from the web emerging from the bath; and

a feeding device for transporting the web material through the first and second baths of acid and sterilant, and the cleaning device.

The apparatus further includes a first recirculation device for feeding the acid solution adjusted to an appropriate concentration back to the first bath by a feedback conduit between an output of the first bath and an input thereof, and a first recirculation bath connected therein, said first recirculation bath having an input for accommodating the introduction of an additional pre-wash chemical.

A second recirculation device for feeding liquid sterilant back to the second bath may also be provided by a feedback conduit between an output of the second bath and an input thereof, and a second recirculation bath in the feedback conduit having an input for accommodating the introduction of additional sterilant.

The pre-wash acids in a preferred embodiment may be citric or acetic acid. The pre-wash station could also contain ozonated water for aseptic applications.

The liquid sterilant may be H₂O₂, ozonated water, or combinations thereof.

The web material for making packages in accordance with the present invention may be formed from plastic, laminates of plastic, flexible foil laminates, combinations of foil and plastic, paper and plastic composite materials, or any other suitable flexible materials.

The packages may be filled with preserved hot or cold sterile liquids, such as citrus juices, low acid beverages, such as milk products, teas, coffees, and particulate material including citrus pulp or fruit particles.

Also provided, in combination with the aforementioned features of the apparatus of the present invention, are pretreatment and post-treatment stations. At the input end there is provided an ionized air treatment station for the web. At the output end, squeegee rolls, hot air knives and an UV irradiation unit are provided to provide post-treatment to the web as it exits the sterilant bath.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic block diagram of an overall form, fill, and seal system in which the sterilizing system of the present invention is utilized;

FIG. 2 is a schematic block diagram depicting the in-line components of the sterilization system and method of the present invention; and

FIG. 3 is a perspective view of a preferred embodiment of the mechanical configuration of the sterilization system of FIG. 2.

### DETAILED DESCRIPTION OF PREFERRED EMBODIMENTS

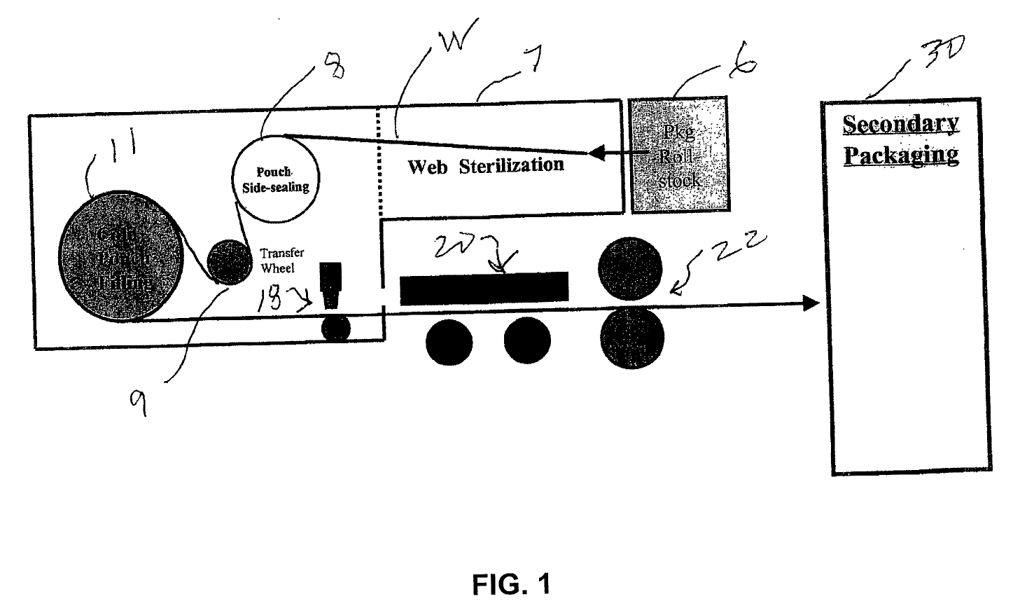

FIG. 1 discloses the package forming and sterilization mechanisms of the system and method of the present invention. The sheet material from which the web W is formed may be provided on a package roll-stock feeder 6. The web W then proceeds through a sterilization mechanism 7. The web W emerges from sterilizer 7, and travels through a pouch side-sealing turret 8, which forms side seams of the pouches P. The web W passes around an idler roller 9 to the perimeter of a cold pouch-filling machine 11. Filled pouches on the web W emerging from filler 11 pass on through a continuous ultrasonic top sealer 18, which seals the top opening of the filled packages. At this point, the packages may pass from the aseptic or ultra-clean environment to merely a clean environment because the packages with sterile liquid or other materials therein have been hermetically sealed. Emerging from sealer 18, web W passes through a straw applicator 20, which may secure a straw to each package. The web W then passes through a die 22 puncture or cutter, and the sealed packages are separated into individual packages for processing by secondary packages 30, which may form cases, multi-paks, and so forth at speeds of about 720 ppm.

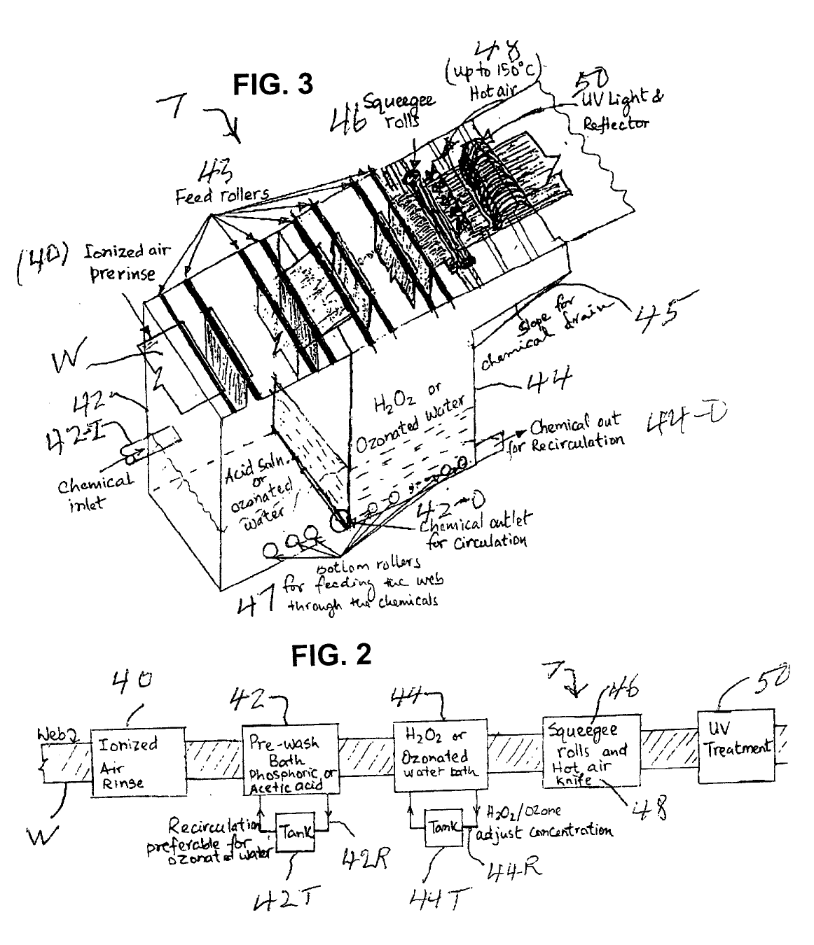

The sterilization system 7 is depicted in more detail in the block diagram of FIG. 2. As illustrated therein, the web W moves from left to right through a series of stations for treating the web. At the input end there is provided an ionized air rinse station 40. Following station 40 is a pre-wash bath 42 containing citric acid or acetic (tartaric, lactic, malic or other organic acids) acid. The pre-wash bath 42 also has a recirculation conduit 42R with a recirculation tank 42T connected therein. Following the pre-wash bath 42, is sterilant bath 44 containing H₂O₂ or ozonated water. Bath 44 also includes a recirculation conduit 44R with a recirculation tank 44R therein. The recirculation tanks, 42T and 44T, are both provided to facilitate adjustment of the concentration of the liquids in the respective baths 42 and 44 during the recirculation phase of those liquids in order to control the efficiency of the washes. Following the bath 44, the web W moves through a series of squeegee rolls 46 and hot air knives 48 to remove excess sterilant and chemicals from the web. As a final treatment of the web W, it passes through a UV irradiation station 50 to further sterilize the web before it proceeds to the form-fill machine of FIG. 1.

Referring in more detail to the system and method illustrated in the block diagram of FIG. 2, at station 40 the ionized air rinse of the web material just before entering the pre-wash bath 42 is effective to prepare the web for treatment in the respective acid and sterilant baths 42 and 44. This ionized air rinse may reduce the microbial load up to 1 log.

The pre-wash bath 42 is used depending on what the product to be packaged is, namely, a preserved ambient fill, or a hot-fill, or aseptic ambient fill. Food grade citric, or acetic acid up to 5% (1-3% preferred), can be used in bath 42 to pre-wash the web at a speed of 1 m/s (might go up to 2 m/s). Other acids such as tartaric, lactic and malic, and water could be used in the pre-wash bath 42. Mechanical washing action at high speeds and the chemicals at 65° C. would help reduce the microbial load by about 1-2 logs. Ozonated water in a recirculation bath 42T could be used for aseptic applications to enhance the sterilization effect. The temperature in the recirculation bath would be up to 30° C., and the concentration is preferably up to 25 ppm (preferred up to 10 ppm). The exposure time within the pre-wash bath is less than 1-2 s, preferably 1 s, but this varies depending on the speed of the web W.

The final wash and sterilant bath 44 in a preferred embodiment is hydrogen peroxide based chemicals (hydrogen peroxide, peroxyacetic acid, etc.). For use for aseptic application, it is preferred that the H₂O₂ concentration is up to 50% at 70° C., and 1-3 seconds exposure time. For different chemicals, the condition will vary. For non-aseptic applications, the pre-wash could utilize acid or water, and the final wash in sterilant bath 44 could have ozonated water. Or in the alternative, the sterilant bath liquid could include a combination of H₂O₂ and ozonated water.

At station 46, excess sterilant is removed by squeegee rolls 46 and hot air knives 48 at temperatures up to 150° C.

At station 50, the web is exposed to ultraviolet (UV) light radiation, of 200-260 nm, or preferably 254 nm from low or medium pressure lamps. The UV treatment may be for up to 3 s exposure (up to 200 mJ/sq cm preferred), but such may be expensive and practically difficult to achieve. So, in any event, the UV treatment should provide at least 100 mJ/sq.cm.

Referring to FIG. 3, which depicts a preferred embodiment of the mechanical configuration of the system depicted in FIG. 2, the stations 40, 42, 44, 46, 50 are shown in-line in the sterilant apparatus 7. Beginning at the left-hand side of the figure, the ionized air pre-rinse 40 is applied to a web W entering the pre-wash bath 42. Pre-wash bath 42 is provided with a chemical inlet 42-l for introducing an acid solution thereinto. Bath 42 is also provided with an outlet 42-O for connection to recirculation conduit 42R and tank 42T of FIG. 2. A series of top-feed rollers 43 and bottom-feed rollers 47 are provided for transporting the web through the liquid in the respective baths 42 and 44 as the web W travels through the sterilizing device to the form, fill, and seal machine. The sterilant bath 44 includes a chemical outlet 44-0 for connection to the recirculation conduit 44R and recirculation tank 44T depicted in FIG. 2. Circulation bath 44 is also provided with an appropriate inlet on the other side of the apparatus depicted in FIG. 3, but that inlet is not shown for clarity purposes. Suitable circulation pumps (not shown) are also provided at appropriate locations.

At the output of bath 44, the squeegee rolls 46 are provided to squeeze excess sterilant and chemicals from the web W. Also provided are hot air knives 48 for further removing excess chemicals from the web. These removed excess chemicals fall into a sloped chemical drain pan 45, and are returned to the sterilant bath 44 for reuse. Also, since the chemical drain pan 45 passes underneath the web W, volatiles from the sterilant flowing through the drain pan contact the overlying web W, and further enhance sterilization thereof. At the output end of the sterilizer 7 of FIG. 3, suitable ultraviolet (UV) light sources, appropriate reflectors, and unit 50 are provided to perform a final sterilizing treatment of the web before it passes to the form, fill, seal machine.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

In the claims:

1. An apparatus for sterilizing web material being fed to a package form-fill-seal machine comprising:
a first bath of liquid chemical for pre-washing the web passing therethrough;
a second bath of liquid sterilant for sterilizing the web passing therethrough;
a cleaning device for removing excess sterilant from the web emerging from the bath; and
a feeding device for transporting the web material through the first and second baths of sterilant, and the cleaning device.

2. The apparatus of claim 1 wherein the chemical is an organic selected from a group consisting of citric acid, acetic acid, tartaric acid, lactic acid, or malic acid.

3. The apparatus of claim 1 wherein the liquid sterilant is ozonated water.

4. The apparatus of claim 1 wherein the liquid sterilant is hydrogen peroxide.

5. The apparatus of claims 1, 2, 3 or 4 further comprising an ultraviolet (UV) radiation source for directing (UV) rays to said web to further sterilize the same.

6. The apparatus of claim 1 further including:
a first recirculation device for feeding excess liquid acid chemical back to said first bath.

7. The apparatus of claim 6 wherein said first recirculation device includes a feedback conduit between an output of the first bath and an input thereof, and a first recirculation bath connected therein, said first recirculation bath having an input for accommodating the introduction of additional pre-wash material.

8. The apparatus of claim 7 wherein the additional pre-wash material is ozonated water.

9. The apparatus of claim 1 further including:
a second recirculation device for feeding liquid sterilant back to said second bath.

10. The apparatus of claim 9 wherein said second recirculating device comprises an inclined drip pan below the web sloping from a point in which the excess sterilant is removed to a point in liquid communication with the bath.

11. The apparatus of claim 9 wherein said second recirculation device includes a feedback conduit between an output of the second bath and an input thereof, and a second recirculation bath having an input for accommodating the introduction of additional sterilant.

12. The apparatus of claim 11 wherein the additional sterilant is ozone.

13. The apparatus of claim 11 wherein the additional sterilant is H₂O₂.

14. The apparatus of claim 11 wherein the additional sterilant is both ozone and H₂O₂.

15. The apparatus of claim 6 further including:
a second recirculation device for feeding liquid sterilant back to said second bath.

16. The apparatus of claim 15 wherein said second recirculation device includes a feedback conduit between an output of the second bath and an input thereof, and a second recirculation bath having an input for accommodating the introduction of additional sterilant.

17. The apparatus of claim 7 further including:
a second recirculation device for feeding liquid sterilant back to said second bath.

18. The apparatus of claim 17 wherein said second recirculation device includes a feedback conduit between an output of the second bath and an input thereof, and a second recirculation bath having an input for accommodating the introduction of additional sterilant.

19. The apparatus of claim 18 further including a device for applying ionized air to the web material prior to entry of the web material into said first bath.

20. The apparatus of claim 1 further including a device for applying ionized air to the web material prior to entry of the web material into said first bath.

21. A method for sterilizing web material being fed to a package form-fill-seal machine comprising the steps of:
providing a first bath of liquid chemical for pre-washing the web passing therethrough;
providing a second bath of liquid sterilant for sterilizing the web passing therethrough;
providing a cleaning device for removing excess sterilant from the web emerging from the bath; and
feeding the web material through the first and second baths of sterilant, and the cleaning device.

22. The method of claim 21 wherein the liquid chemical is an organic acid selected from a group consisting of citric acid, acetic acid, tartaric acid, lactic acid, or malic acid.

23. The method of claim 21 wherein the liquid sterilant is ozonated water.

24. The method of claim 21 wherein the liquid sterilant is hydrogen peroxide.

25. The method of claims 21, 22, 23 or 24 comprising the further step of:
directing (UV) rays to said web to further sterilize the same.

26. The method of claim 21 further including the step of:
feeding excess liquid chemical back to said first bath.

27. The method of claim 26 including the further steps of:
providing a feedback conduit between an output of the first bath and an input thereof, and a first recirculation bath connected therein, and introducing additional pre-wash material into the recirculation bath.

28. The method of claim 27 wherein the additional pre-wash material is ozonated water.

29. The method of claim 21 further including the step of:
feeding liquid sterilant back to said second bath.

30. The method of claim 29 including the step of:
providing an inclined drip pan below the web sloping from a point in which the excess sterilant is removed to a point in liquid communication with the bath.

31. The method of claim 29 including the further step of:
providing a feedback conduit between an output of the second bath and an input thereof, and a second recirculation bath and introducing additional sterilant into the second recirculation bath.

32. The method of claim 31 wherein the additional sterilant is ozone.

33. The method of claim 31 wherein the additional sterilant is H₂O₂.

34. The method of claim 31 wherein the additional sterilant is both ozone and H₂O₂.

35. The method of claim 28 further including the step of applying ionized air to the web material prior to entry of the web material into said first bath.

36. The method of claim 21 further including the step of applying ionized air to the web material prior to entry of the web material into said first bath.

37. An apparatus for sterilizing web material being fed to a package form-fill-seal machine comprising:
a bath of liquid sterilant for sterilizing the web passing therethrough;
a feeding device for transporting the web material through the bath of sterilant;
a cleaning device for removing excess sterilant from the web emerging from the bath; and
a recirculating device for feeding the excess sterilant back to said bath through a path open to an underside of the web material emerging from the bath to thereby expose the web to any volatiles of the sterilant in said path.

38. The apparatus of claim 37 wherein the liquid sterilant is ozonated water.

39. The apparatus of claim 37 wherein the liquid sterilant is hydrogen peroxide.

40. The apparatus of claims 37, 38 or 39 further comprising an ultraviolet (UV) radiation source for directing (UV) rays to said web to further sterilize the same.

41. The apparatus of claims 37, 38 or 39 wherein said recirculating device comprises an inclined drip pan below the web sloping from a point in which the excess sterilant is removed to a point in liquid communication with the bath.
